(19) United States
(12) Patent Application Publication
(10) Pub. No.: US 2023/0031392 A1
(43) Pub. Date: Feb. 2, 2023

(54) KIT OF DENTAL INSTRUMENTS FOR INTRAORAL SCANNING

(71) Applicant: SALUS ORIS S.R.L., Vallesaccarda (AV) (IT)

(72) Inventor: Domenico LO RUSSO, Vallesaccarda (AV) (IT)

(21) Appl. No.: 17/788,107

(22) PCT Filed: Dec. 10, 2020
(86) PCT No.: PCT/IB2020/061747

§ 371 Date: Jun. 22, 2022

(30) Foreign Application Priority Data
Dec. 23, 2019 (IT) 102019000025249

(51) Int. Cl. A61B 1/247; A61B 1/00; A61C 17/10; A61C 9/00

(52) U.S. Cl. CPC A61B 1/247; A61B 1/00172; A61C 9/004; A61C 17/10

(57) ABSTRACT

A kit of dental instruments for intraoral scanning includes first instrument and second instruments for a patient’s left and right side lower arches, respectively, at least a third instrument for the upper arch. The first instrument includes a first handle with rectangular profile and rounded edges and two arms, the latter being a first left lateral arm on whose body a scanner slides, and a second front arm, the arms being extractible and interchangeable. The first lateral arm inserts in the handle through holes, the second instrument being the mirrored copy of the first instrument and including a second handle analogous to the first handle and with rounded edges and two arms, the latter being a first right lateral arm and a second front arm analogous to the second front arm for the left side, the kit further including at least a third instrument for the upper mouth arch.

### CROSS-REFERENCE TO RELATED TO PRIORITY APPLICATIONS

This application is the U.S. national phase of International Application No. PCT/IB2020/061747 filed Dec. 10, 2020 which designated the U.S. and claims priority to IT Patent Application No. 102019000025249 filed Dec. 23, 2019, the entire contents of each of which are hereby incorporated by reference.

### BACKGROUND OF THE INVENTION

### Field of the Invention

The present description refers to the medical field and in particular to the dental field. Still more in detail, the present invention regards a particular kit of dental instruments to be used for the intraoral scanning of arches, typically but not exclusively, edentulous of a patient.

### Description of the Related Art

At present, the removable prostheses, used for the rehabilitation of people who are missing some or all of their teeth, are constructed starting from impressions of the arches of the patient. Such impressions are made with materials of various type, which are usually prepared by mixing a base compound and a catalyst; just prepared, these impression materials have a pasty consistency and require specific trays (instruments adapted to contain them) so they can be inserted in the mouth of the patient in contact with the arches where, by hardening, they take on the shape thereof, registering it. From this impression, which is the negative of the three-dimensional shape of the arches, a model will be created (which represents the positive thereof, i.e. it has the same shape as the arches). Such model is obtained by casting, in the impression, a material which is usually a specific plaster, which in turn must solidify so that it can be used. This first model cannot be used for constructing the prosthesis since it is not suitably accurate.

Such lack of accuracy depends on the fact that the materials used for the relief of the impression undergo size modifications that are proportional to the quantity and to the thickness of the material used. Since the first impression is detected/measured by using a standard tray, which as such has average dimensions so to be applied in all mouths, this inevitably involves an approximation that requires the use of great quantities of material, especially of nonuniform thicknesses; increased size modifications ensue and, even worse, there are nonuniformities in the different zones of the arch, which determine distortions and inaccuracies such to render the first model, derived from that impression, not sufficiently accurate for the construction of the prosthesis. Therefore, the first model is only used for constructing an individual tray for that patient, so as to be able to detect/measure a second impression with a minimum quantity of material and of uniform thicknesses. All this clearly involves the need to repeat the procedure, with clear negative aspects such as: expenditure of time for the patient, from the patient’s standpoint, repeating the same procedure signifies having to be present at multiple operating sessions; this can constitute an important problem, especially if one considers that in general people who need removable prostheses are elderly people, who live alone and often and not completely self-sufficient and can have difficulties in moving from their home or from the residential structures that house them in order to reach the dentist’s office.

Irritation/discomfort for the patient can also be encountered. The relief of the impression is quite unpleasant due to the sensation of suffocation that it generally determines and/or due to the stimulation of the vomit reflex, to which many are particularly sensitive, also in relation to the bad taste of many of the impression materials that require nearly five minutes for a correct hardening in the mouth of the patient. In addition to all this, there are problems relative to potential allergy phenomena with respect to the materials used.

Another disadvantage is represented by the expenditure of time for the clinic and for the dental laboratory. This negatively conditions the efficiency of the health structures and of the relative laboratories, inevitably causing an increase of the costs which in the end negatively affects the user of the service, the patient, and sometimes negatively affects the possibility of access to treatment.

Also the expenditure of materials has multiple implications, tied to the expenditure of resources for the acquisition of such materials, as well as for the management thereof, and with regard to the disposal thereof with consequent environmental impact.

In recent years, new technologies have been introduced in dentistry, some of which allowing the detection of the shape of the teeth and gums of the patient by means of actual intraoral scanners. Such intraoral scanners are commonly used for making the fixed dental prosthesis, i.e. that anchored to the teeth, but are not used for the removable prosthesis, especially for the removable total prosthesis (that for the patient who has no residual tooth remaining). The reason for such lack of use is tied to the fact that, in the situation in which teeth are missing and actually only the alveolar arches covered by gums and mucous membrane remain, it is extremely complex to detect an intraoral scanning. This is tied to the fact that the surface to be scanned (i.e. that of the gums and of the mucous membrane which covers the edentulous arches) lacks geometric characteristics that are easily detectable by the scanner and, above all, it is movable. In practice, and in an extremely simplistic manner, the intraoral scanner detects a series of three-dimensional images of the object to be subjected to scanning and unites them, one after the other, in order to compose the object; if the object to be scanned (in our case the surface of the edentulous mucous membrane) is moved, it is clear that it is impossible, or in any case extremely inaccurate, to reconstruct a shape which is changed during the scanning process.

The objective of the present invention is to overcome such difficulty, by simplifying and making it possible to predictably obtain the intraoral scanning of alveolar arches that are totally or partially edentulous.

### SUMMARY OF THE INVENTION

The present description refers to a particular instrument for medical use and specifically for dental use, which allows attaining a precise intraoral scanning of the edentulous alveolar arches. All this in order to achieve, with greater precision, the dental prosthesis and also speed up the procedure of prosthetic implant in the patient. More specifically, according to the present invention, an auxiliary instrument was implemented which allows attaining the following objectives:
Spreading open the tissues surrounding the edentulous arches. With the loss of teeth, the bone (alveolar arch) that supported such teeth is reabsorbed, its size being reduced and being moved downward for the lower arch and upward for the upper arch. Following this, the cheeks and, for the lower arch, the tongue tend to be superimposed on the edentulous crest and to mask it. Therefore, for a correct scanning of the edentulous arches it is necessary to spread open, i.e. physically move, and independent of the patient’s will, the cheeks and the tongue so as to move them away from the arches themselves and expose them in their completeness so as to prevent zones where the cheeks and the tongue are superimposed or “by placing in a shadow” the edentulous arch, which would prevent a full reading and capture thereof by the scanner.
Stabilizing and holding still the tissues. As detailed above, a fundamental characteristic of the edentulous arches is that the mucous membrane which covers them is movable. This is tied to the fact that as the edentulous arches are progressively reabsorbed and their size reduced, the gum adhered thereto is partially lost, so that with covering of the edentulous arches, a movable mucous membrane remains whose level of mobility is generally proportional to the reabsorption of the alveolar arches. More in detail, “movable” signifies that the musculature of cheeks and tongue which takes the insertion close to the alveolar crests, being contracted can move the mucous membrane that covers the edentulous alveolar arches. This also signifies that small involuntary movements of the patient can, as described above, prevent the scanning of the arches that are totally or partially edentulous. Therefore, for the purpose of a correct intraoral scanning of the edentulous arches, it is imperative to hold still and stabilize the tissues of the arch for the entire duration of the scanning process.
Removing the saliva and preventing the obfuscation of the scanner. During the scanning process, above all in the lower arch, saliva will tend to be accumulated; this could compromise the correct scanning of several zones, since the current scanners are in the end based on optical technologies that use the reflection of the light, which could be affected by the different refraction index of the saliva. Therefore, a great advantage is achieved by removing the excess saliva during the scanning process and with the same instrument. The presence of suction also allows preventing the mirror present on the tip of the scanner from being obfuscated due to the air breathed out by the patient.
Constituting a guide for the scanner. The possibility of maintaining the tip of the scanner (that which actually captures the object) always at an optimal distance from the surface to be scanned constitutes a great advantage since it facilitates the focusing of the object and hence the acquisition of the images and consequently their accuracy and processing. For such reason, the instrument, object of the present invention, is attained in a manner so as to allow the tip of the scanner to be rested on its parts and slide thereon, like a guide capable on one hand of simplifying the moving of the tip of the scanner during the scanning process and on the other hand of allowing the maintenance of an optimal distance from the surface to be captured. In addition, such characteristic is useful for allowing an automated scanning in association with an extraoral device that supports the present guide object and, on the basis thereof, simultaneously support and imparts a movement adapted for the scanner.

The instrument created — with its capacity of spreading open the tissues, stabilizing them and keeping them still for the entire duration of the scanning process, removing the saliva, guiding and allowing a more fluid movement of the tip of the scanner and keeping the latter at an optimal distance from the edentulous crest - allows enabling a quick, accurate and predictable intraoral scanning of the arches that are totally or partially edentulous. Therefore, it can have application in various fields and disciplines of dental prosthesis and odontostomatology; in particular, the preferred fields provide for the use as:
removable total prosthesis with mucous support
removable partial prosthesis with mucous support
removable total prosthesis with implant support
removable partial prosthesis with implant support
prosthesis on implants
fixed prosthesis on natural teeth with extended edentulous saddles.

Said instrument specifically allows the acquisition and documentation of oral anatomic data of the patient for diagnostic, anthropometric or identification purposes. Its use is also provided for in patients with natural teeth in which the involuntary movements of the oral musculature prevent a correct scanning of the dental arches and in the poorly-collaborating patients with natural teeth (only by means of example: patients with cognitive impairment of various nature).

Its use is also contemplated in all possible odontostomatological fields, not expressly mentioned, in which it is necessary to reach the above objectives for which the proposed instrument can be useful and therefore have suitable application. In detail, for the removable prothesis applications, the use of the instrument makes possible its application thereto and their intraoral scanning, allowing preventing the conventional impression path, i.e. two conventional impressions and the construction of the individual tray and of two plaster models, since the intraoral scanning is itself the work model that is sufficiently accurate for the construction of the prosthesis.

In other words, this allows obtaining a series of multiple advantages and benefits for the patient consisting of: preventing different clinical sessions; reducing the times necessary for treatments; simplifying the organizational aspect with regard to elderly people who may require assistance in order to reach the dentist’s office; and preventing unpleasant or otherwise risky procedures like those tied to convention impression. In addition, the intraoral scanning is mucostatic (captures and acquires the shape of the oral mucous membrane as is without touching nor compressing it, contrary to the action of conventional impression materials, which inevitably exert a certain pressure on the mucous tissues):

regarding such characteristic, it was demonstrated that it is possible to obtain final prostheses that better reflect the tissues of the patient. All this reducing the size of the future reabsorption of the edentulous arches.

Also for the dental office, various advantages are recognizable: improving the efficiency; avoiding costs relative to the impression materials; avoiding the expenditure of time for processing that is not requested; the intraoral scanning, once executed, is constituted by a digital file that can be moved between the dental office and the production laboratory through telecommunication systems that are nearly instantaneous and do not require the mailing of physical impressions or models; improving the standardization of the procedures.

Further advantages are for the benefit of citizens in general, such as: improving and facilitating the access to treatments; greater care for the environment (no materials to dispose of); the impressions and the physical models are in contact with the fluids of the oral cavity which are potentially infected and infective: even if they are suitably decontaminated, their circulation, as well as the final disposal, in any case involves a minimum risk which can, with the intraoral scanning, be entirely canceled.

### BRIEF DESCRIPTION OF THE DRAWINGS

The invention will be described hereinbelow in detail with reference to the enclosed figures in which:

FIG. 1 shows a perspective view of the first instrument 1 for the left side lower arch of the patient of the kit according to the present invention. FIG. 1(a) shows an upper view thereof. FIG. 1(b) shows a front view thereof and FIG. 1(c) shows a side view and in particular the angle of divergence of the lateral arm with respect to the vertical plane.

FIG. 2 shows a perspective view of the pair of instruments for the edentulous lower arch and in particular FIG. 2(a) shows the second instrument 10 for an edentulous lower arch, right side of the patient and which is the mirrored copy of the first instrument 1 for an edentulous lower arch left side of the patient (FIG. 2(b)).

FIG. 3 shows the travels that the tip of the scanner is made to undergo and the respective sequence indicated with arrows indicated with letters A, B, C, D.

FIG. 4 shows a plan view of the third instrument 100 for the upper arch. FIG. 4(a) shows its upper view; FIG. 4(b) shows its frontal view; FIG. 4(c) shows its side view with indication of the angle of divergence of the lateral arms with respect to the vertical plan.

FIG. 5 shows a plan view of the third instrument 100 for the upper arch and in particular the scanning strategy for the edentulous upper arch. More in detail, in said figure, the travels that the tip of the scanner is made to undergo and the respective sequence are indicated with the arrows A′, B′, C′, D′.

### DESCRIPTION OF THE PREFERRED EMBODIMENTS

In all embodiments thereof and in order to attain specific objectives of the present invention, and in consideration of the anatomic characteristics of the upper and lower edentulous arches of the patient, the dental instruments of the present kit have a shape that is optimized and specific for the upper arch and a different shape for the lower arch; in particular, for the lower arch, two mirrored shapes are necessary, one for the right side and one for the left side. This particular assembly allows providing one portion suitable for the upper arch, one for the right side of the lower arch and one for the left side of the lower arch.

Finally, the object of the present description is a kit of instruments 1, 10, 100 respectively for: the left side lower arch of the patient, the right side lower arch of the patient, and the upper arch.

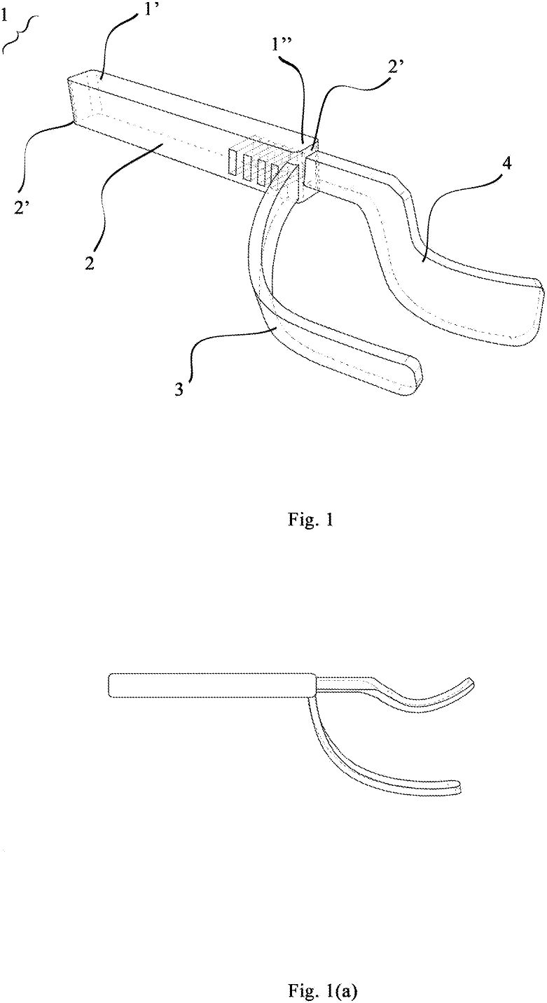

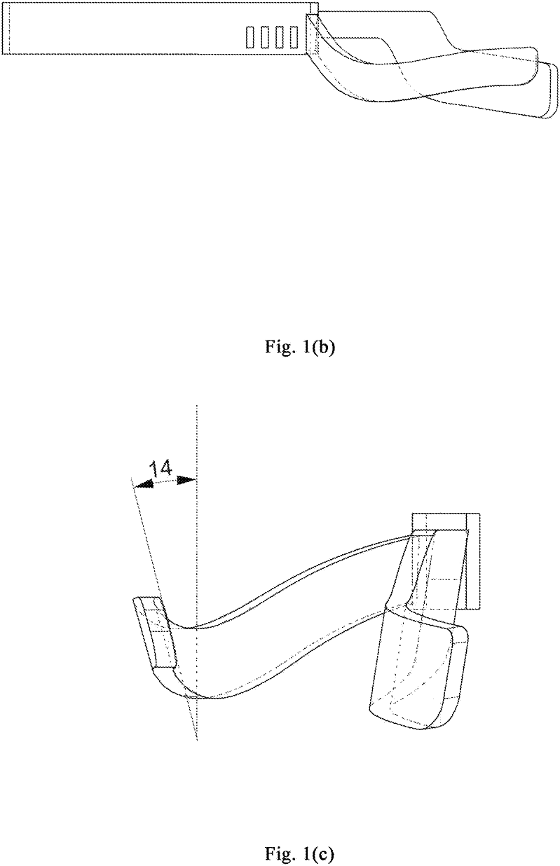

With reference to FIG. 1, and in particular to FIG. 1(a), the first instrument 1 for the left side lower arch of the patient comprises: a first handle 2 with parallelepiped shape and with slightly rounded edges 2', at one of whose ends, first end 1' and second end 1", and in particular on said second end 1", two arms are applied: a first left lateral arm 3 (the lateral arm) directed outward which serves for spreading open the cheek, and on which the body of the tip of the scanner can be rested and made to slide, and another arm, the second arm 4, i.e. the front arm, directed in front direction as if it was the extension of the handle, and which serves to support and control the tongue and simultaneously to provide a stop at the end of the tip of the scanner. Both said arms can be extracted so as to allow the interchangeability with the arms of the opposite side, as well as with arms of different size, necessary for the adaptation to the individual variability between the patients. Said first left lateral arm 3 is inserted in the handle by means of through holes with section typically but not exclusively rectangular, which allow the alternating insertion of the right or left arm, each entering the hole from its own side. On said handle 2, in the direction of its length, at least one, preferably at least two and still more preferably a plurality of holes are present for connecting said second lateral arm 3, i.e. so as to allow modifying the distance between the arms, a characteristic which optimizes the use in relation to the width of the edentulous arch. Both said second lateral arm 3 and second front arm for the left front side 4, in order to be better adapted to the anatomy of the patient, are suitably shaped in the three spatial dimensions according to the schemes reported in FIGS. 1(a), (1b) and 1(c).

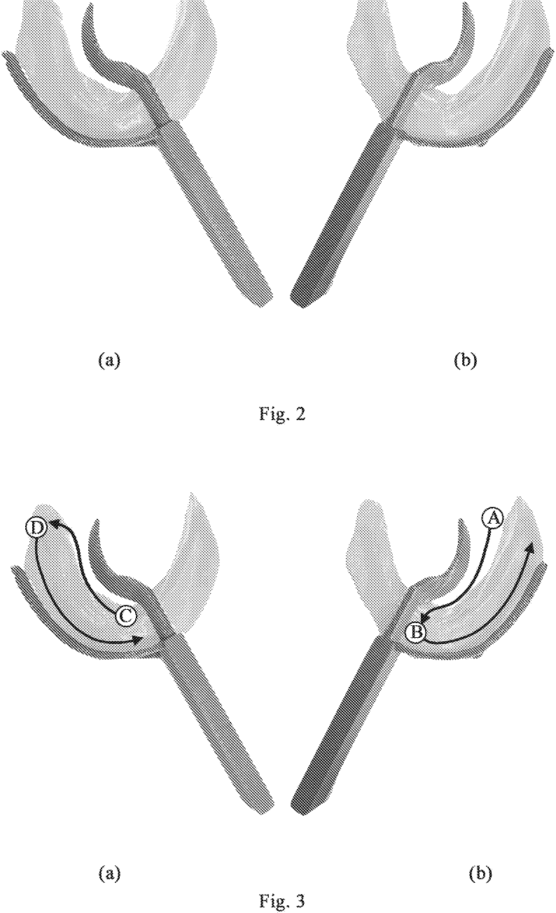

The second instrument 10 for the right side lower arch of the patient is the mirrored copy of that of the left side of the patient, i.e. of the aforesaid first instrument 1. Thus, with reference to FIG. 2 and in particular to FIG. 2(a), said second instrument 10 for the right side lower arch of the patient likewise comprises a second handle 20, analogous to said first handle 2, with rounded edges 20' and a pair of arms, i.e. a first lateral arm for the right lower arch 30 and a second front arm for the right side 40 analogous to the second front arm for the left side 4 of said first instrument 1.

The pair of said first instrument 1 for the left side lower arch and second instrument 10 for the right side lower arch, in addition to allowing the spreading of the tissues, their retraction and stabilization during the scanning process, constitutes an actual guide aimed to simplify and facilitate the movement of the tip of the scanner. Indeed, during the scanning process, the tip of the scanner can be physically rested on the arms that constitute the instrument and it can be simply moved thereon, like on a track, with a fluid movement and without being obstructed, also enabling the maintenance of an optimal distance from the tissues.

All this facilitates the scanning process and allows maintaining the acquisition tip of the scanner always in the range of the depth of field and focus of the scanner, which improves and also simplifies the stitching process, i.e. for software processing and joining of the 3D images acquired for the purpose of reconstructing the shape of the surface subjected to scanning. Therefore the kit of dental instruments has been designed in a manner such to guide and support the intraoral scanning, integrating and incorporating the most suitable scanning strategies therein. In other words, the strategies of intraoral scanning, i.e. the mode with which the intraoral scanning is executed and the path that the tip of the scanner must follow so to be able to acquire the edentulous arches with accuracy and in their entirety, have been developed, elaborated and tested together with the kit, and the shape of its instruments has been optimized so as to facilitate and support the preferred execution of such scanning strategies.

Such scanning strategies for the edentulous lower arch are reported in FIG. 3. In practice, the intraoral scanning of the edentulous lower arch starts from the left side of the patient. The acquisition starts from the posterior region (i.e. left retromolar trigone of the patient), by keeping the tip of the scanner moved lingually with respect to the center of the crest and slightly rotated towards the lingual slope of the crest itself so as to optimize the orientation thereof towards the surface to be acquired; simultaneously, the tip of the scanner touches the front arm of the instrument, which prevents it from being moved away from the crest, and rests on the lateral arm that supports it. The tip of the scanner is thus moved in a manner such that by sliding along the surface of the front arm of the instrument, it can complete the travel indicated by the arrow A in FIG. 3, which allows acquiring the entire occlusal-lingual side of the edentulous crest.

Once the path indicated with the arrow A has been completed, the acquisition continues by moving the tip of the scanner according to the travel indicated with the arrow B, keeping it rested on the lateral arm of the instrument.

Once this is done, the instrument for the left side is removed and the instrument for the right side of the patient is introduced and positioned.

The acquisition starts again from the median region, by keeping the tip of the scanner moved lingually with respect to the center of the crest and slightly rotated toward the lingual slope of the crest itself in a manner so as to optimize the orientation thereof towards the surface to be acquired; simultaneously, the tip of the scanner touches the front arm of the instrument, which prevents it from being moved away from the crest, and rests on the lateral arm that supports it. The tip of the scanner is then moved in a manner such that by sliding along the surface of the front arm of the instrument, it can complete the travel indicated by the arrow C (FIG. 3). Once the posterior region has been reached (i.e. right retromolar trigone of the patient), the acquisition continues, by moving the tip of the scanner according to the travel indicated with the arrow D, keeping it rested on the lateral arm of the instrument.

Such scanning sequence is optimal for the right-handed operators, while for left-handed operators it is preferable to reverse, in a mirrored manner, the sequences reported in FIG. 3. With regard to the edentulous upper arch, said kit can, as stated above, further comprise a third instrument 100 for the edentulous upper arch. For the latter, given its anatomic shape, it is thus possible to make the intraoral scanning by using only one instrument as aid.

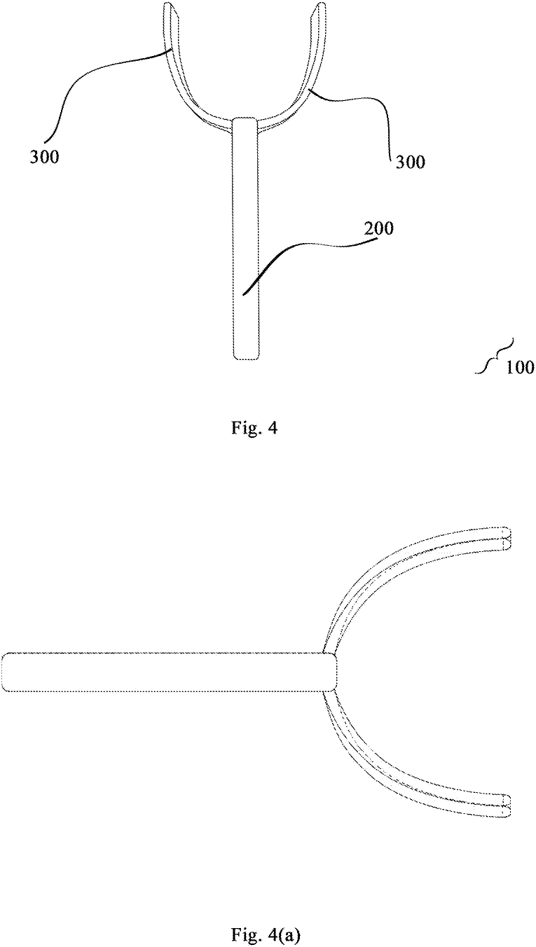

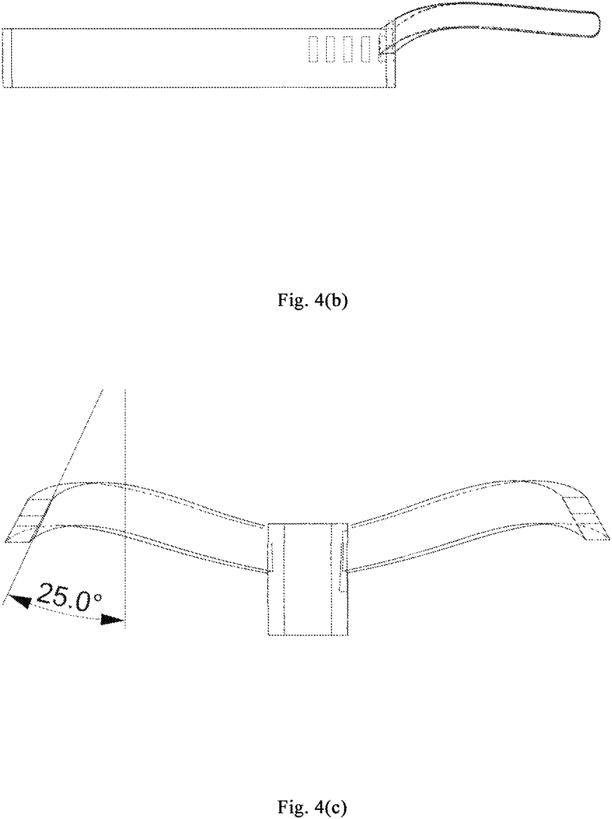

With reference to FIG. 4, the aid instrument for the intraoral scanning of the upper arch comprises a third handle 200 with parallelepiped shape with slightly rounded edges 200', at one of whose ends two arms 300 being applied that are both directed outward, respectively towards the right and towards the left; such arms serve for spreading open the cheeks, and the tip of the scanner can be rested and made to slide on such arms.

With reference to FIG. 4, it is indicated that both said symmetric arms 300 are extractible so as to allow the interchangeability with arms of different sizes, necessary for the adaptation to the individual variability between the patients. The symmetric arms 300 are inserted in the handle by means of through holes with rectangular section that allow the insertion of the right and left arm, each entering a hole from its own side. On the handle, in the direction of its length, different holes are present for connecting the lateral arms.

The symmetric arms, in order to be better adapted to the anatomy, are suitably shaped in the three spatial dimensions.

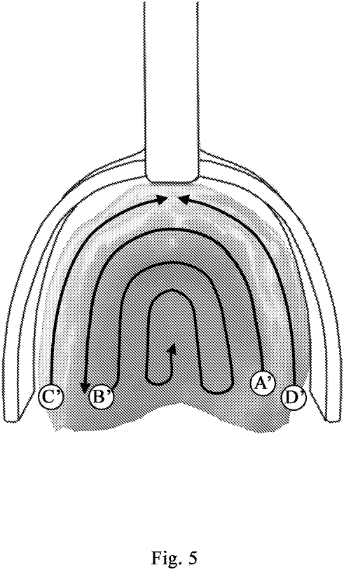

Said third instrument 100 of the kit, object of the present invention, in addition to allowing the spreading open of the tissues, their retraction and stabilization during the scanning process, constitutes an actual guide aimed to simplify and facilitate the movement of the tip of the scanner. Indeed, during the scanning process, the tip of the scanner can be physically rested on the symmetric arms 300 and it can be simply moved thereon, like on a track, with a fluid movement and without being obstructed, also enabling the maintenance of an optimal distance between the tissues. All this facilitates the scanning process and allows maintaining the acquisition tip of the scanner always in the range of the depth of field and focus of the scanner, which also improves and simplifies the stitching process, i.e. for software processing and joining of the 3D images acquired for the purpose of reconstructing the shape of the surface subjected to scanning. Therefore, said third instrument 300 was designed in a manner such to guide and support the intraoral scanning, integrating and incorporating the most suitable scanning strategies. In other words, the strategies of intraoral scanning, i.e. the mode with which the intraoral scanning is executed and the path that the tip of the scanner must follow so to be able to acquire the edentulous arches with accuracy and in their entirety, have been developed, elaborated and tested together with the instrument. Such scanning strategies for the edentulous upper arch are reported in FIG. 5 which shows the travels that the tip of the scanner is made to complete and the respective sequence, and which are indicated with the arrows.

In practice, the intraoral scanning of the edentulous upper arch starts from the left side of the patient. The acquisition starts from the posterior region (i.e. left maxillary tuberosity of the patient) by keeping the tip of the scanner at the center of the crest. The tip of the scanner is moved by following the crest center, in a manner such to complete the travel indicated with the arrow A′ (FIG. 5).

After having completed the path indicated with the arrow A′, the acquisition continues by moving the tip of the scanner according to the travel indicated with the arrow B′, which will allow acquiring the palate in its entirety.

Once this is done, the acquisition restarts from the region of the right maxillary tuberosity of the patient, by keeping the tip of the scanner moved vestibularly with respect to the center of the crest and slightly rotated towards the vestibular slope of the crest itself in a manner so as to optimize the orientation thereof towards the surface to be acquired; simultaneously, the tip of the scanner rests on the lateral arm of the instrument which supports it and guides it. The tip of the scanner is thus moved, in a manner such that by sliding along the surface of the arm of the instrument it can complete the travel indicated with the arrow C′ (FIG. 5) and acquire the external side of the edentulous crest up to the bottom of the corresponding arch. Once the median region is reached, the acquisition is interrupted in order to start again from the region of the left maxillary tuberosity of the patient, by undergoing the travel indicated with the arrow D′ and with the same modes described for the travel C′.

The kit according to the present invention makes use of the aid of, or it can further comprise in some preferred embodiments thereof, an apparatus for the automatic guiding of the scanner during the intraoral scanning.

Such apparatus consists of a body within which a through cavity is present, similar to the hole of a doughnut; such cavity constitutes the housing for the intraoral scanner which is inserted therein on one side such that the tip of the scanner exits from the opposite side. Such housing, in addition to supporting the scanner, is movable in the three spatial directions, thus allowing the movement of the scanner.

At the side where the tip of the scanner exits outward, the housings are present for the handles of the above-indicated guide instruments. Such holes are coordinated with the housing of the scanner in a manner such that the tip of the scanner can make contact with the arms of the corresponding guide instruments (in a manner analogous to when the scanner is managed manually). The data coming from the sensors on the guide instruments, as well as the data deriving from the gyroscope of the scanner, can be suitably integrated and processed in order to allow the unit for controlling the movement of the housing of the scanner to make the latter carry out the movement necessary for completing the scanning.

In its preferred embodiment, said kit comprises at least a first handle 2, or at least a second handle 20, or at least a third handle 200 and arms shaped for the various arches: i.e. at least a first left lateral arm 3 and first right lateral arm 30 for the left and right lower arch, respectively, and at least a second front arm left side 4 or second front arm right side 40. At least two symmetric arms 300 for the edentulous upper arch are also comprised. Thus, all the shapes, i.e. those for the upper arch and two (right and left) for the lower arch, share the same handle. Reference can be reported on the arms which indicate the scanning direction.

The variant for left-handed operators is provided only for the instruments relative to the lower arch and differs from the standard version due to the fact that the indication on the arms of the instruments, regarding the paths relative to the scanning strategy, has been reversed.

In addition, the left arm of the instrument relative to the upper arch can be attained in a variant that provides for a small rear extension, that allows the arm to be inserted in the hole of the handle subsequent to the hole there the right arm is housed, in order to allow the same identical position of the free end of the two arms, in the direction of the length.

An attachment mechanism can also be attained for attaching the two arms, right and left, in a manner such that both engage a single hole for the insertion in the handle.

Both for the instrument relative to the upper arch and for those relative to the lower arch, a child version is provided for, in which the relative arms have length and height reduced by 35%. The handle, as well as the relative mechanisms for connecting between the handle and the arms, remain unchanged.

In addition, several embodiments provide that the front arm has been traversed by a tunnel-shaped cavity which opens with 2 mm diameter holes on the lower side thereof. Such cavity continues with an analogous cavity present in the handle and the aim thereof is to be connected to the terminal of a sucker so to be able to carry out the suction and removal of the saliva during the use of the instrument.

A further version provides that the lateral arm and the internal arm are provided with sensors for the tip of the scanner such to assist the automated movement unit in producing a fluid and controlled movement.

Preferably, but not exclusively, in all embodiments thereof, said kit of dental instruments is such that its instruments have the following dimensions: said first instrument 1 for the left side lower arch of the patient and second instrument 10 for the right side lower arch of the patient have total length comprised between 150 mm and 154 mm, preferably is 152 mm; the first left arm 3 of the patient and the first right lateral arm have length comprised between 55 and 71 mm, preferably is 63 mm; said first front arm 4 for the left side and second front arm 40 for the right side have length comprised between 62 and 68 mm, preferably is 66 mm. Said third instrument 100 is such that its arms 300 have length comprised between 45 and 60 mm, preferably is 55 mm and that the distance between the free ends of said arms is comprised between 58 and 70 mm, and preferably is 64 mm.

1. Kit of dental instruments for intraoral scanning, comprising at least a first instrument for the left side lower arch of the patient, at least a second instrument for the right side lower arch of the patient, at least a third instrument for the upper arch of the patient, said first instrument comprising a first handle with rectangular profile and with rounded edges and two arms, the two arms being at least a first left lateral arm, on whose body a scanner is made to slide, and at least a second front arm, said arms being extractible and interchangeable, said first lateral arm being insertable in the handle by means of a plurality of holes, said second instrument being the mirrored copy of said first instrument and comprising a second handle analogous to said first handle and with rounded edges and a pair of arms, the pair of arms being a first right lateral arm and a second front arm analogous to the second front arm for the left side, said kit further comprising at least a third instrument for the upper mouth arch comprising a third handle with parallelepiped shape with slightly rounded edges, at one of whose ends two arms are applied, both directed outward respectively towards the right and left side of the mouth apparatus of the patient, said arms being adapted to spread open the cheeks and allow the sliding of a scanner on the body thereof.

2. The kit according to claim 1, wherein the holes in which the arms are inserted in an interchangeable manner — on said first handle, second handle and third handle - have rectangular profile.

3. The kit according to claim 1, wherein said first instrument for the left side lower arch of the patient and second instrument for the right side lower arch of the patient have total length comprised between 150 mm and 154 mm and wherein the first left arm of the patient and the first right lateral arm have length comprised between 55 and 71 mm, while said first front arm for the left side and second front arm for the right side have length comprised between 62 and 68 mm.

4. The kit of dental instruments according to claim 3, wherein said first instrument for the left side lower arch of the patient and second instrument for the right side lower arch of the patient have total length of 152 mm and wherein the first left lateral arm of the patient and the first right lateral arm have length of 63 mm, while said first front arm for the left side and second front arm for the right side have length of 66 mm.

5. The kit of dental instruments according to claim 1, wherein the right and left lateral arms of the third instrument for the upper mouth arch have length comprised between 45 mm and 60 mm and wherein the distance between the free ends of said arms is comprised between 58 mm and 70 mm.

6. The kit of dental instruments according to claim 5, wherein the right and left lateral arms of the third instrument for the upper mouth arch have length of 55 mm and wherein the distance between the free ends of said arms is 64 mm.

7. The kit of dental instruments according to claim 1, wherein for said third instrument, an attachment mechanism is provided for attaching the right and left arms, which provides for the insertion of said arms in a single hole for the insertion in the handle.

8. The kit of dental instruments according to claim 1, wherein on each of the arms of each of said first instrument, second instrument and third instrument, indicator signals are present that indicate the scanning direction of a scanner adapted to execute the scanning of the arches of the mouth apparatus of the patient.

9. The kit of dental instruments according to claim 1, wherein the signals indicating the scanning direction of the scanner are reversed in the case of left-handed operators.

10. The kit of dental instruments according to claim 1, wherein for the instrument relative to the upper arch as well as for those relative to the lower arch, a child version is provided for, in which the relative arms have length and height reduced by 35%.

11. The kit according to claim 1, wherein at least a front arm for the left side lower arch or arm for the right side lower arch is traversed by a tunnel-shaped cavity which opens with 2 mm diameter holes on the lower side thereof, said cavity being adapted to be connected to the terminal of a saliva sucker.

12. The kit according to claim 1, wherein at least an arm of the instruments of said kit is provided with sensors for the tip of a scanner.

13. The kit according to claim 1, wherein an apparatus is further comprised for the automatic guiding of the scanner for the intraoral scanning.

14. The kit of dental instruments according to any claim 1, suitable for use in the treatment of mouth apparatuses of patients with edentulous arches.

15. The kit according to claim 2, wherein said first instrument for the left side lower arch of the patient and second instrument for the right side lower arch of the patient have total length comprised between 150 mm and 154 mm and wherein the first left arm of the patient and the first right lateral arm have length comprised between 55 and 71 mm, while said first front arm for the left side and second front arm for the right side have length comprised between 62 and 68 mm.

16. The kit of dental instruments according to claim 2, wherein the right and left lateral arms of the third instrument for the upper mouth arch have length comprised between 45 mm and 60 mm and wherein the distance between the free ends of said arms is comprised between 58 mm and 70 mm.

17. The kit of dental instruments according to claim 3, wherein the right and left lateral arms of the third instrument for the upper mouth arch have length comprised between 45 mm and 60 mm and wherein the distance between the free ends of said arms is comprised between 58 mm and 70 mm.

18. The kit of dental instruments according to claim 4, wherein the right and left lateral arms of the third instrument for the upper mouth arch have length comprised between 45 mm and 60 mm and wherein the distance between the free ends of said arms is comprised between 58 mm and 70 mm.

19. The kit of dental instruments according to claim 2, wherein for said third instrument, an attachment mechanism is provided for attaching the right and left arms, which provides for the insertion of said arms in a single hole for the insertion in the handle.

20. The kit of dental instruments according to claim 3, wherein for said third instrument, an attachment mechanism is provided for attaching the right and left arms, which provides for the insertion of said arms in a single hole for the insertion in the handle.
